(19) United States
(12) Plant Patent Application Publication
(10) Pub. No.: US 2026/0033414 P1
(43) Pub. Date: Jan. 29, 2026

(54) DIANTHUS PLANT NAMED 'KLEDP23382'

(50) ;

(71) Applicant: KLEMM + SOHN GMBH & CO. KG, Stuttgart (DE)

(72) Inventor: Lena Thiede, Riederich (DE)

(73) Assignee: KLEMM + SOHN GMBH & CO. KG, Stuttgart (DE)

(21) Appl. No.: 18/781,821

(22) Filed: Jul. 23, 2024

(51) Int. Cl. A01H 6/30; A01H 5/02

(52) U.S. Cl. CPC A01H 6/305; A01H 5/02
USPC PLT/276

(57) ABSTRACT

A new Dianthus plant designated ‘KLEDP23382’ particularly distinguished by pink flowers with a medium, upright habit, is disclosed.

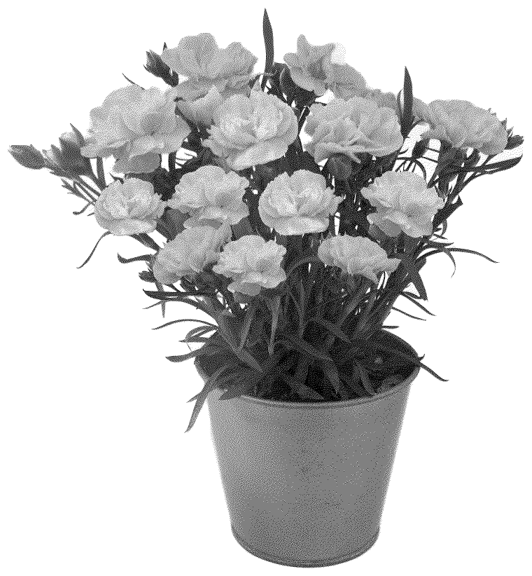

Genus and species: Dianthus caryophyllus L.

Variety denomination: 0‘KLEDP23382’.

### BACKGROUND OF THE NEW PLANT

The present invention comprises a new and distinct variety of Dianthus, botanically known as Dianthus caryophyllus L. and hereinafter referred to by the variety name ‘KLEDP23382’. ‘KLEDP23382’ originated from a cross conducted in the summer of 2015 in Stuttgart, Germany between the proprietary Dianthus female parent ‘KLEDP13121’ (unpatented) and the proprietary Dianthus male parent ‘DSS-2014-1017’ (unpatented). The seeds from this cross were sown in Stuttgart, Germany and a single plant was selected in 2015.

In August 2015, ‘KLEDP23382’ was first vegetatively propagated by vegetative stem cuttings in Stuttgart, Germany. ‘KLEDP23382’ was found to reproduce true to type in successive generations of asexual propagation in Stuttgart, Germany via vegetative cuttings.

### SUMMARY

The following are the most outstanding and distinguishing characteristics of ‘KLEDP23382’ when grown under normal horticultural practices in Stuttgart, Germany.
1. Pink flowers; and
2. A medium vigor; and
3. Upright habit.

### DESCRIPTION OF THE PHOTOGRAPH

This new Dianthus plant is illustrated by the accompanying photograph of a 13-week-old plant taken in April 2022 and grown in a greenhouse in Stuttgart, Germany. The colors shown are as true as can be reasonably obtained by conventional photographic procedures. The photograph is a whole plant photo of ‘KLEDP23382’ and shows the foliage, buds, and flowers.

### DESCRIPTION

The following detailed descriptions set forth the distinctive characteristics of ‘KLEDP23382’. The data which define these characteristics were collected from asexual reproductions carried out in Stuttgart, Germany. Data was collected in April 2024 from plants that were planted in week 37 in 2023 and grown in a greenhouse. Color references are to The R.H.S. Colour Chart of The Royal Horticultural Society of London (R.H.S.), 5^{th} edition (2007).
Classification:
   Family.—Caryophyllaceae.
   Botanical.—Dianthus caryophyllus L.
   Designation.—‘KLEDP23382’.
   Type.—Pot carnation.
Parentage:
   Female parent.—Dianthus ‘KLEDP13121’ (unpatented).
   Male parent.—Dianthus ‘DSS-2014-1017’ (unpatented).
Plant:
   Propagation type.—Vegetative cuttings.
   Time to produce a rooted young plant.—28 days.
   Root description.—Fine roots, RHS 155C.
   Rooting habit shape.—Lanceolate.
   Plant form.—Upright.
   Plant height.—24.0 cm.
   Plant diameter or spread.—28.0 cm.
Lateral branches/stems:
   Shape of the branch in cross-section.—Circular.
   Hollowness of the branch.—Absent.
   Length.—22.0 cm.
   Diameter.—26.0 cm.
   Internode length.—3.8 cm.
   Internode quantity per branch.—4.
   Number of internodes per branch/stem.—4.
   Node length.—0.5 cm.
   Node diameter.—0.6 cm.
   Texture.—Smooth.
   Color.—RHS N137A.
   Flower clustering on the lateral branch.—In all lateral branches.
Foliage:
   Arrangement.—Opposite.
   Length.—6.4 cm.
   Width.—0.6 cm.
   Shape.—Obovate.
   Apex.—Acute.
   Base.—Obtuse.
   Margin.—Entire.
   Texture (both upper and lower surfaces).—Smooth.
   Cross-section of the leaf.—Moderately concave.
   Curvature of the leaf.—Absent.
   Glaucosity of the leaf.—Weak.
   Leaf color (both immature and mature).—Immature: Upper surface: RHS 147A. Lower surface: RHS 137A. Mature: Upper surface: RHS 147A. Lower surface: RHS 137A.
   Venation pattern.—Absent.
Flowers:
   Flower type and habit.—Double sprays.
   Fragrance.—Present.
   Position of the flowers compared to the foliage of the plant.—Moderately above.
   Profile of both the upper and lower part in the lateral view.—Flat.
   Diameter of the flower.—5.1 cm.
   Natural flowering season.—Spring to autumn.
   Number of flowers per spray.—2.
   Flower longevity on the plant.—10 days.
Buds:
   Length.—2.3 cm.
   Diameter.—1.2 cm.
   Shape.—Obovate.
   Color.—RHS 137B.
   Extrusion of the styles at the bud stage.—Absent.
Corolla:
   Quantity of petals and arrangement.—36 and arranged in 4 whorls.
   Corolla height.—2.4 cm.
   Petal length.—3.8 cm.
   Petal width.—1.9 cm.
   Petal shape.—Obtuse.
   Petal apex.—Obtuse.
   Petal base.—Acute.
   Petal margin.—Crenate.
   Depth of petal margin.—Shallow.
   Petal texture (both upper and lower surfaces).—Smooth.
   Petal color.—Immature: Upper surface: RHS 65A. Lower surface: RHS 69C. Mature: Upper surface: RHS 73C. Lower surface: RHS 69C.
   Macule.—Present; color is RHS N155B.
Calyx:
   Quantity of sepals and arrangement.—Composed of 4 sepals.
   Longitudinal axis of the lobes of the calyx.—Straight.
   Shape.—Campanulate.
   Calyx height.—2.4 cm.
   Calyx diameter (at the apex).—1.0 cm.
   Sepal texture (both upper and lower surfaces).—Smooth.
   Sepal color.—Upper surface: RHS 137B. Lower surface: RHS 145A.
   Position of the outer lobes of the epicalyx in relation to the calyx.—Adpressed.
   Apex shape of the outer lobes of the epicalyx.—Acute.
   Tip length of the outer lobes of the epicalyx.—0.2 cm.
   Apex shape of the inner lobes of the epicalyx.—Short acuminate.
   Tip length of the inner lobes of the epicalyx.—0.7 cm.
Peduncles:
   Length.—0.3 cm.
   Diameter.—0.2 cm.
   Strength.—Strong.
   Texture.—Smooth.
   Color.—RHS 137A.
Reproductive organs:
   Stamens.—Quantity per flower: 4. Filament length: 0.9 cm. Filament color: RHS 145D. Anther length: 0.5 cm. Anther diameter: 0.1 cm. Anther color: RHS N155A. Pollen amount: None observed.
   Pistils.—Quantity per flower: 1. Length: 1.5 cm. Diameter: 0.6 cm. Stigma: Color: RHS NN155A. Shape: Filamentous. Length: 0.2 cm. Diameter: Less than 0.1 cm.
   Style.—Color: RHS NN155A. Length: 0.2 cm. Diameter: Less than 0.1 cm.
   Ovary.—Color: RHS 145A. Width: 0.5 cm.
Seeds: None observed to date.
Disease and insect resistance: No particular disease or insect resistance observed.

### COMPARISON OF ‘KLEDP23382’ WITH PARENTAL LINES AND KNOWN VARIETY

‘KLEDP23382’ is distinguished from the parents as described in Table 1.

TABLE 1
Comparison with Parental Lines

| Characteristic | ‘KLEDP23382’ | Female parent | Male parent |
|---|---|---|---|
| Flower color | Pink | Light pink | Dark red |

‘KLEDP23382’ can be compared to the commercial Dianthus variety ‘KLEDP07089’ (unpatented). Differences between the varieties are described in Table 2.

TABLE 2
Comparison with Similar Variety

| Characteristic | ‘KLEDP23382’ | ‘KLEDP07089’ |
|---|---|---|
| Flower color | Pink | Pink and purple |
| Habit | Medium vigor | Compact |
| | with upright | |
| | habit | |

I claim:

1. A new and distinct variety of Dianthus plant designated ‘KLEDP23382’ as illustrated and described herein.
